(19) United States
(12) Patent Application Publication
(10) Pub. No.: US 2017/0340355 A1
(43) Pub. Date: Nov. 30, 2017

(54) DEVICE TO ADJUST A STRIP OF CIRCULAR ADJUSTMENT AROUND THE CEPHALIC POLE

(71) Applicant: AIR BAG ONE SARL, LUXEMBOURG (LU)

(72) Inventor: Jorge Ernesto ODON, BANFIELD (AR)

(73) Assignee: AIR BAG ONE SARL, LUXEMBOURG (LU)

(21) Appl. No.: 15/527,209

(22) PCT Filed: May 19, 2016
(86) PCT No.: PCT/EP2016/061343

§ 371 Date: May 16, 2017

(30) Foreign Application Priority Data
May 20, 2015 (AR) 20150101565

(51) Int. Cl. A61B 17/44

(52) U.S. Cl. CPC A61B 17/442

(57) ABSTRACT

A device to adjust a strip of circular adjustment around the cephalic pole, that is inserted within the delivery channel together with a sleeve and an applicator. The sleeve is flexible, has both ends open, and a fold in its middle where it is fixed, on the inner side of the fold, and where the sleeve has two traction handles on ends of the inner fold, and where the applicator is provided with a butt head, spatulas that fit in the pockets of the sleeve and a guide to indicate the movement of the spatulas with respect to the head butt, and includes: a strip of circular adjustment of flexible material; and at least an adjustment axis with its respective driving mechanism. Also disclosed is a method of using the device.

### BACKGROUND

The present invention relates to a device to adjust a strip of adjustment to the cephalic pole of a fetus during delivery and its method of use, more precisely to aid in the crowning of the fetus' head.

### PREVIOUS ART

At present, there exists a plurality of media to hold the head of a fetus during the delivery.

The ‘forceps’ (vacuum device) is one of them. The forceps is an obstetric instrument having the form of tongs, that is used to help in the extraction of the fetus from the exterior. The disadvantage of said instrument is that it causes: lesions in the soft tissue of the mother's pelvis, in particular the cervix and the anal sphincter; it may leave temporary marks on the head or on the face of the neonate; lesions in the brachial plexus of the neonate. Fractures on the fetus' skull are rarely frequent.

U.S. Pat. No. 1,782,814 of Eugene Froehlich of Nov. 25, 1930 discloses an “obstetric extractor”. This extractor consists of a structure made of soft, deformable and flexible material to be placed surrounding the fetus' head and comprises a shrinkable section for the reception of the head and a traction section. In order to reduce the section to receive the head, there are ropes which, by pulling them, the opening where the fetus' head is located, is closed.

U.S. Pat. No. 4,875,482 of Harari, dated Oct. 24, 1987, discloses a “flexible grasping device”. This device consists of a cylindrical net that surrounds the fetus' head and presents, in its distal end, a ring that closes the net by pulling from a cord.

WO patent application 9317629 of Gary Alexander, published on Sep. 16, 1993, discloses a “device for assistance during a delivery”. This device comprises a hollow, extended and flexible member that fits around the fetus' head. A fixed collar is provided at the end of the side of the head of the fetus, so as to restrict the opening of said end.

U.S. Pat. No. 5,559,413 of Gary Alexander, dated Jan. 14, 1997, discloses a “device for assistance during a delivery”. This device comprises a hollow, extended and flexible member with at least an open end that fits around the fetus' head. A means of restriction on said end is provided to reduce the size of the opening of said end.

U.S. Pat. No. 5,910,146 of Gary Alexander, dated Jun. 8, 1999, discloses a “device for assistance during a delivery”. This device comprises a hollow, extended and flexible member with at least an open end that fits around the fetus' head and an adjustable rope.

Argentine patent application 20070103245, published under AR 062010 A3 on Oct. 8, 2008 discloses a “device to extract elements contained within a cavity”. This device comprises a folded sleeve provided with an air chamber on said fold, which is inserted around the fetus' head. When the air chamber is inflated, same is adjusted around the fetus' head, to bind it.

U.S. Pat. No. 6,398,790 of Gary Alexander, dated Jun. 4, 1992, discloses a “device for assistance during a delivery”. This device comprises separated sheets of loops made of polypropilene which are woven loose, where each sheet has a control end, an opposed opening and a pair of insertion rods. The sheets are inserted one after the other, being connected through the insertion rods, until they completely surround the head of the fetus. An adjustable cord is provided.

US patent application 20040015175 of Nguyen, published on Jan. 22, 2004, discloses a “pipe of air in the shape of a glove, forceps and is empty to open the delivery channel during childbirth”. This device comprises a glove of double stationary wrapper, which coverage to fingers leaves at least the first phalanx uncovered. The hand with the glove is inserted within the vagina and is inflated, the ends of the uncovered fingers hold the head of the fetus.

The cited antecedents provide areas of contact of device-head relatively small, whether because they are linear in circle or point-like as the ones of Nguyen. Argentine patent application 20070103245 discloses an area of greater contact, but not of the size originally expected.

### SUMMARY

The present invention consists of a device comprised by a strip of circular adjustment and a means to reduce said strip, wherein the strip of circular adjustment is fit loosely around the cephalic pole, and with the turn of the means to reduce the strip, a smooth adjustment is obtained on the cephalic pole. The strip of circular adjustment has the shape of a truncated cone or a truncated cone with a circular portion at the greater diameter. The adjustment is made through at least one rotatory means.

The method consists in adjusting, in one or more adjustments, the strip of circular adjustment around the cephalic pole, rotating at least one rotation means.

### Objects of the Present Invention: Device

The object of the present invention is to provide a device to adjust the strip of circular adjustment around the cephalic pole, to be useful for different sizes of cephalic poles.

Another object of the present invention is to provide a device to adjust a strip of circular adjustment around the cephalic pole which needs not to be regulated during the extraction of the fetus.

Another object of the present invention is to provide a device to adjust a strip of circular adjustment around the cephalic pole and that provides less opposition during the traction, reducing the risks of causing damages to the mother's soft parts until the crowning.

Another object of the present invention is to provide a device to adjust a strip of circular adjustment around the cephalic pole with less possibilities of obstructing the fetus' arteries.

Another object of the present invention is to provide a device to adjust a strip of circular adjustment around the cephalic pole, which facilitates the insertion of same within the delivery channel, closing the ends of the tentacles of the applicator.

Another object of the present invention is to provide a device to adjust the strip of circular adjustment around the cephalic pole, to indicate the size of said cephalic pole.

Another object of the present invention is to provide a device to adjust a strip of circular adjustment around the cephalic pole that presents a greater effectiveness in the traction, so that same does not get out of the device.

Another object of the present invention is to provide a device to adjust a strip of circular adjustment around the cephalic pole that facilitates the task of the operator, so as not to be pendent of different parameters.

Another object of the present invention is to provide a device to adjust a strip of circular adjustment around the cephalic pole that allows the rotation or not of the fetus within the delivery channel.

### Objects of the Present Invention

### Method

Another object of the present invention is to disclose a method for the insertion of the device so that the tips of the spatulas do not exert pressure against the wall of the delivery channel.

Another object of the present invention is to disclose a method that manages to determine the size of the cephalic pole.

Another object of the present invention is to disclose a method that allows to rotate or leave the fetus fixed within the delivery channel.

### DESCRIPTION OF THE FIGURES

FIG. 1 shows a perspective view of the device to adjust a strip of circular adjustment around the cephalic pole.

FIG. 2 shows the rotating effect of the insertion rod over the strip of circular adjustment, seen from the front on the smaller side of same.

FIG. 3 show a lateral view of two embodiments of strips of circular adjustment.

FIG. 4 shows a lateral view of the device installed in an extended sleeve.

FIG. 5 shows a perspective view of the device installed in a sleeve in the way of two axises of adjustment, close one to the other.

FIG. 6 shows an applicator.

FIG. 7 shows the steps of the method of usage, where, due to clarity purposes, the folded sleeve was left out.

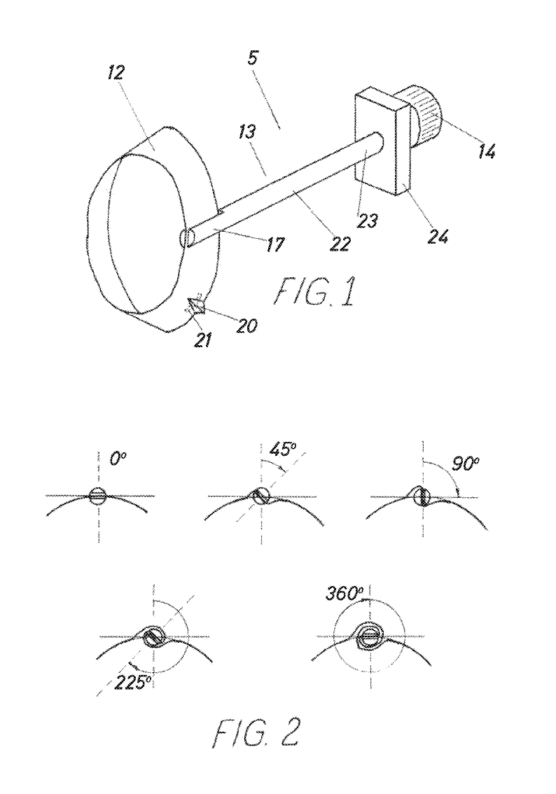

FIG. 1 shows the components of the device where the strip of circular adjustment passes by a longitudinal cut of the end of the rod and the driving mechanism is direct, and a pinch over said strip of circular adjustment.

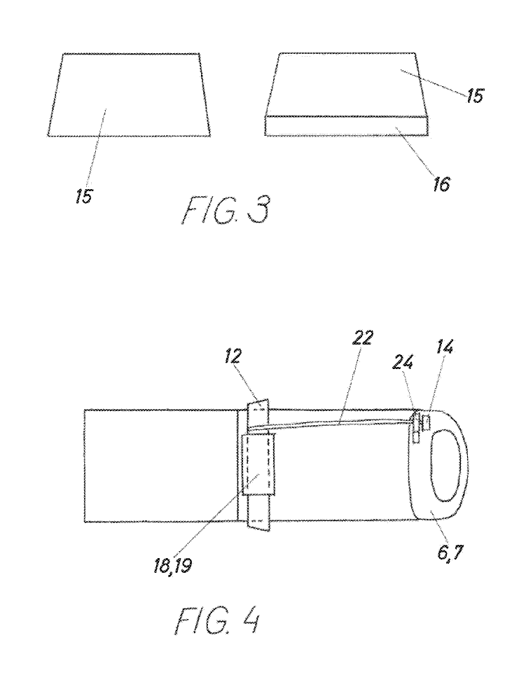

FIG. 2 shows the rotational effect of the rod at different angles: 0°, 45°, 90°, 225° and 360°.

FIG. 3 shows a view of the strip of circular adjustment with the shape of a truncated cone and a view of the strip of circular adjustment with the shape of a truncated cone with a portion of circumference in the greater diameter.

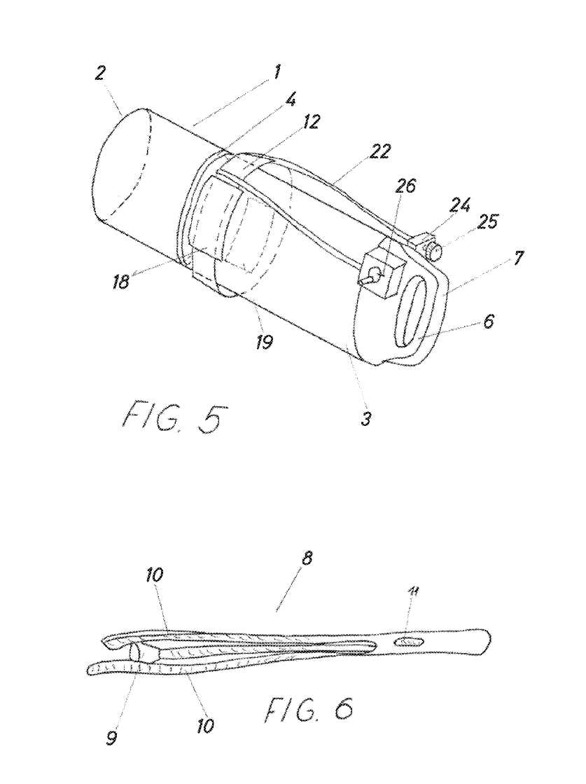

FIG. 4 shows a lateral view of the device installed in an extended sleeve where the driving mechanism is direct and is fixed to a traction handle of the sleeve.

FIG. 5 is a perspective view of the device installed in a sleeve in the way of two axises of adjustment, close one to the other, where the driving mechanism is in the same plane as of that of the rod and the other is in a different plane as of that of the rod.

FIG. 6 shows the applicator where the top of the head, the spatulas and the guide indicating the movement are seen.

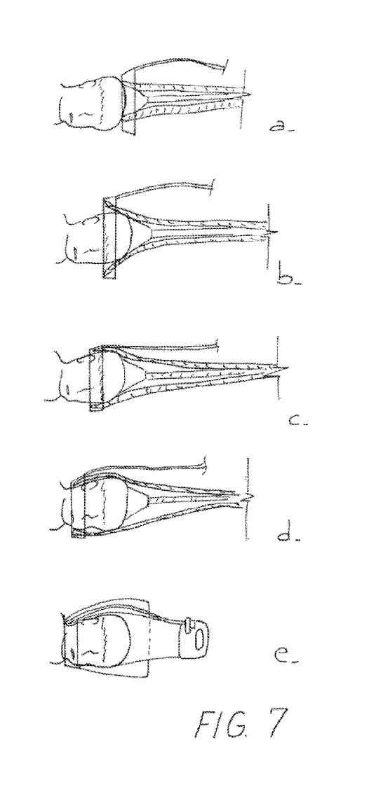

FIG. 7 shows the steps of the method of usage, where, due to clarity purposes, the folded sleeve was left out, where:

a) the device-sleeve-applicator set is inserted within the delivery channel until the indicating guide marks the middle of the travel made by the spatulas;
b) the first adjustment to the strip of circular adjustment is made by rotating at least one adjustment axis;
c) on the indicator of the rotation angle, the diameter of the head of the fetus is read;
d) the device-sleeve-applicator set is continued to be inserted until its final position;
e) it is continued with a final adjustment of the strip of circular adjustment of at least half turn (the fitted sleeve is shown); and
the applicator is removed.

### DESCRIPTION OF THE INVENTION

The device to adjust a strip of circular adjustment around the cephalic pole is used together with two elements known in the previous art:
A flexible sleeve (1), with both ends open (2,3), with a fold (4) in its middle part where, on the external side of the inner fold, the device (5) is fixed to adjust a strip of circular adjustment around the cephalic pole. The sleeve provides longitudinal pockets in the inner part of the internal fold to lodge the applicator spatulas and at least two traction handles (6,7) in the ends of the internal fold, and
An applicator (8) for the insertion of the flexible sleeve folded around the fold, with the device to adjust a strip of circular adjustment around the cephalic pole. The applicator provides a head butt (9), spatulas (10) that fit in the longitudinal pockets of the sleeve and a guide (11) indicating the movement of the spatulas in respect to the head butt.

### Strip of Circular Adjustment

The device to adjust a strip of circular adjustment around the cephalic pole comprises a strip of circular adjustment (12) and at least an adjustment axis (13) with its respective driving mechanism (14).

The strip of circular adjustment is a truncated cone (15) or a truncated cone with a circular portion (16) in the bigger diameter of the cone. It is made of a flexible material, to which the distal ends (17) of at least one adjustment axis are fixed. The material may be reusable.

Strips of circular adjustment with different diameters may be provided for cases of fetuses with different sizes of cephalic poles. The tie between the strip of circular adjustment and the ends of the adjustment axis is such that, when said adjustment axises rotate, the strip of adjustment is wound around the adjustment axis, reducing the diameter of the strip of circular adjustment.

The turn may be made in both senses. In the case of two adjustment axises, these may be fixed one close to the other or on positions diametrically opposed.

The surface of contact between the element that fastens the cephalic pole and said cephalic pole is important because it will determine the adequate pressure that the latter will receive. The greater the surface of contact is, the lesser pressure the cephalic pole will receive to pull it up to its crowning.

The surface of contact of the strip of circular adjustment of the present invention is substantially greater than in other devices known in the previous art. In these, the surface of contact is a line of greater or lesser thickness around the cephalic pole, generally around the neck and nape, where attention is to be given so as not to strangle the fetus too much if the device was inserted too much. In our invention, by being a circular strip of a considerable width and by having the shape of the trunk of a cone, said strip is anatomically adjusted around the cephalic pole resulting in a surface of contact substantially greater, also favored by the tilted shape that helps in the holding of the cephalic pole. It never holds only the neck, since given the height of the adjustment strip, although it is supported on the fetus' shoulders, there will always be a portion of the adjustment strip that will coincide with the chin, maxillaries and mouse of the fetus.

The sleeve provides on the external side of the inner fold, some media (18,19) to fix the strip of circular adjustment in a loose manner. Preferably, they are open supports of transversal orientation as to the sleeve, whereby said strip of circular adjustment is provided.

The complete unwinding of the strip of circular adjustment may be aided from the distal end of the rod with elastic strips so that said unwinding be quicker and with less tangles. If small pinches (20) are formed over the bigger diameter of the truncated cone and their basis are tied with an elastic strip (21), the portion of the bigger diameter of the truncated cone will tend to be more closed (less diameter) when not in operative position, than when it is adjusted around the cephalic pole. These pinches would be placed away from the union of the distal end of the rod with the strip of circular adjustment, in a way that when the tension over the strip of circular adjustment is released, the elastic strips will drive forming the pinch and will reduce the tangle of the strip of adjustment around the distal end of the rod.

### Adjustment Axis

Each adjustment axis comprises a rod (22) with a distal end fixed to the strip of circular adjustment, and the proximal end (23) fixed to a driving mechanism of said rod. The driving mechanism may be fixed, by means of a support (24), to the traction handles of the sleeve. Materials that compose same may be reusable.

The driving mechanism causes the rotation of the adjustment axis so that it may reduce the diameter of the strip of circular adjustment winding same around the rod. The turn may be made in any sense, clockwise or counterclockwise.

The driving may be direct, where the rotation angle of the driving mechanism is equal to the rotation angle of the rod, or indirect where, by means of reductions, the rotation angle of the driving mechanism corresponds with the partial rotation angle, preferably smaller, of the rod. The advantage of the latter modality is that a greater precision in the adjustment may be obtained.

The rod is made of a flexible material with enough stiffness so as not to get bent.

The driving mechanism may be provided with an indicator of the rotation sense (25), so that it indicates in which sense, clockwise or counterclockwise, the rod was rotated. In one embodiment, the numbers or scales have different color whether the rotation has been made clockwise or counterclockwise.

The driving mechanism may be also provided with an indicator of the rotation angle (26), to measure the degrees the rod rotated starting from an angle θ, for example when the rod does not have any wound portion of the strip of circular adjustment. By rotating the rod, the angle (in degrees) in which the rod rotated may be measured. This measure has its correlation, through the relation of reduction and external diameter of the distal end of the rod, which is variable as the strip of circular adjustment is wound, with the diameter of the cephalic pole. The rotation indicator may also measure the rotation angle of the driving mechanism.

In one embodiment, blocks may be provided so that the rod does not get released and some mechanism of automatic release to quickly free the rod.

### Method

The method to use the device of the present invention consists of partially inserting the device-sleeve-applicator set within the delivery channel, then make a first adjustment of the strip of circular adjustment, complete the insertion until its full entrance, make the final adjustment of the strip of circular adjustment and remove the applicator.

The partial insertion of the set within the delivery channel is made until the butt of the applicator head makes contact with the cephalic pole, when the spatulas keep being introduced within the delivery channel around the cephalic pole, while the butt of the applicator head gets fixed on the cephalic pole. This makes that the indicating guide moves until a mark to indicate that the middle movement of the spatulas has been reached. At that time, the ends of the spatulas are in proximity of the bigger diameter of the cephalic pole. By making the first adjustment of the strip of circular adjustment, the size of the cephalic pole may be measured on the indicator of the rotation angle or its equivalent in the diameter of the cephalic pole.

The insertion of the device-sleeve-applicator set is continued until its final position is indicated on the indicating guide. This second insertion results easier because the ends of the spatulas do not now point outwards, towards the walls of the delivery channel, but in parallel direction, for they have been adjusted by the strip of circular adjustment. Lacerations of said wall are reduced. A final adjustment is made on the strip of circular adjustment so that it is not dragged when removing the applicator.

The applicator is removed leaving the device in position and the sleeve folded.

Any additional adjustment that is made, will make the fetus rotate, by dragging, in the same direction of the rotation of the rod.

To make up for this effect, which sometimes is desired, a second adjustment axis is provided, which would rotate in the opposite sense of the first adjustment axis to make up for both draggings.

These steps are clearly shown in FIG. 5:
Step a) the device-sleeve-applicator set is inserted until butting the fetus' head, when the distal ends of the spatulas begin to open. The strip of circular adjustment is loose within the sleeve;
Step b) shows when the ends of the spatulas reached the maximum size of the cephalic pole, the strip of circular adjustment being still loose;
Step c) shows how the ends of the spatulas remain after the adjustment of the strip of circular adjustment. At this instance, the size of the diameter of the head may be read on a corresponding reader. It can be seen that the ends of the spatulas do no longer point outwards but in parallel manner as to the applicator axis;
Step d) shows the set in its final position with the strip of circular adjustment in its final adjustment; and
Step e) shows the situation of the previous step once the applicator is removed.

As previously described, the adjustment of the strip of circular adjustment is made rotating one or the adjustment axises. Trials made with special dummies have shown that in order to adjust the strip of circular adjustment rotating only one adjustment axis turns the cephalic pole. For this reason, the preferred embodiment is of two adjustment axises that rotate in opposite sense so as to make up for the rotation of the cephalic pole. Anyway, it is known that on many occasions, it is necessary or convenient to cause said rotation. In these cases, the person who attends the birth inserts his hand within the vagina, holds the cephalic pole and makes it rotate. With our invention, this rotation is managed rotating only one of the adjustment axises.

1. A device to adjust a strip of circular adjustment around the cephalic pole, that is inserted within the delivery channel together with a sleeve and an applicator,
wherein the sleeve is a flexible sleeve, which has both ends open, with a fold in its middle where it is fixed, on the inner side of the fold, and the sleeve is provided with at least two traction handles in the ends of the inner fold, and
where the applicator is provided with a head butt, spatulas that fit in the pockets of the sleeve and a guide to indicate the movement of the spatulas with respect to the head butt.
wherein it comprises:
a strip of circular adjustment of flexible material; and
at least an adjustment axis with its respective driving mechanism.

2. The device to adjust a strip of circular adjustment around the cephalic pole of claim 1, wherein the strip of circular adjustment has the shape of the trunk of a cone or the trunk of a cone with a cylindric portion on the bigger diameter.

3. The device to adjust a strip of circular adjustment around the cephalic pole of claim 1, wherein the strip of circular adjustment is fixed loosely to the sleeve through open supports of transversal orientation.

4. The device to adjust a strip of circular adjustment around the cephalic pole of claim 1, wherein each adjustment axis comprises a rod with a distal end and a proximal end with a driving mechanism in said proximal end.

5. The device to adjust a strip of circular adjustment around the cephalic pole of claim 1, wherein the distal end of the rod is adjusted to the strip of adjustment so that, when rotating the rod, the strip of circular adjustment is wound around the distal end of said rod.

6. The device to adjust a strip of circular adjustment around the cephalic pole of claim 1, wherein in the embodiment of two adjustment axises, these are provided one in proximity of the other or diametrically opposed.

7. The device to adjust a strip of circular adjustment around the cephalic pole of claim 1, wherein the driving mechanism is tied directly with the rod of the adjustment axis.

8. The device to adjust a strip of circular adjustment around the cephalic pole of claim 1, wherein the driving mechanism is tied through a reduction mechanism with the rod of the adjustment axis.

9. The device to adjust a strip of circular adjustment around the cephalic pole of claim 1, wherein the driving mechanism is on a different plane than that of the rod.

10. The device to adjust a strip of circular adjustment around the cephalic pole of claim 1, wherein an indicator of the rotation sense is provided.

11. The device to adjust a strip of circular adjustment around the cephalic pole of claim 1, wherein an indicator of the rotation angle is provided.

12. The device to adjust a strip of circular adjustment around the cephalic pole of claim 11, wherein said indicator of rotation angle also indicates the size of the diameter of the adjustment strip, equal to the diameter of the cephalic pole.

13. The device to adjust a strip of circular adjustment around the cephalic pole of claim 1, wherein the driving mechanism is fixed to one of the traction handles of the sleeve.

14. The device to adjust a strip of circular adjustment around the cephalic pole of claim 1, wherein the materials with which they are made of are reusable.

15. The device to adjust a strip of circular adjustment around the cephalic pole of claim 1, wherein at least one pinch is provided in the area of biggest diameter of the cone and its basis are tied by an elastic strip that tends to keep said basis of the pinch together.

16. A method of usage of the device to adjust a strip of circular adjustment around the cephalic pole of claim 1, wherein it comprises the steps of:
(a) Inserting the device-sleeve-applicator set within the delivery channel until the indicating guide marks the middle of the travel made by the spatulas;
(b) Making the first adjustment to the strip of circular adjustment by rotating at least one adjustment axis;
(c) reading on the indicator of the rotation angle, the diameter of the head of the fetus;
(d) continuing to insert the device-sleeve-applicator set until its final position, shown by the indicating guide;
e) continuing with the final adjustment of the strip of circular adjustment of at least half turn;
f) removing the applicator; and
g) pulling from the sleeve's handles to extract the fetus.

17. The method of usage of the device to adjust a strip of circular adjustment around the cephalic pole of claim 16, wherein it also comprises the step of adjusting at least the two adjustment axises in opposite sense to avoid the rotation of the cephalic pole after the step of removing the applicator.
